(19) United States
(12) Patent Application Publication
(10) Pub. No.: US 2014/0243523 A1
(43) Pub. Date: Aug. 28, 2014

(54) HUMAN EPITHELIAL CELL LINE FOR 3-D MODELIZING OF CANCER AND TREATMENT THEREOF

(71) Applicants: CNRS, Paris (FR); GALDERMA RESEARCH & DEVELOPMENT, Biot (FR)

(72) Inventors: Elodie Burty, Sourcieux-les-Mines (FR); Thierry Magnaldo, Nice (FR); Yannick Gache, Nice (FR); Jérôme Aubert, Grasse (FR)

(73) Assignees: GALDERMA RESEARCH & DEVELOPMENT, Biot (FR); CNRS, Paris (FR)

(21) Appl. No.: 14/346,911

(22) PCT Filed: Sep. 24, 2012
(86) PCT No.: PCT/EP2012/068778

§ 371 Date: Mar. 24, 2014

Related U.S. Application Data
(60) Provisional application No. 61/538,602, filed on Sep. 23, 2011.

(51) Int. Cl. G01N 33/50

(52) U.S. Cl. CPC G01N 33/5017
USPC 544/118; 435/366; 435/29; 435/7.1

(57) ABSTRACT

A cellular model is described that targets dysregulation or inappropriate activation of the Sonic Hedgehog/Patched (SHH/PTCH) pathway. Also described, is a screening method using this cellular model to screen for pharmacological compounds that can treat or prevent skin cancer, in particular, Basal Cell Carcinoma (BCC) lesions.

The present invention is in the domain of pharmacy and more specifically in skin cancer area and particular for Basal Cell Carcinoma (BCC). The present invention provides a cellular model targeting the Sonic Hedgehog/Patched (SHH/PTCH) pathway dysregulation or inappropriately activated as well as screening method using this cellular model to screen pharmacological compounds able to treat or prevent BCC lesions.

The Hedgehog pathway is normally active during embryonic development and plays a central role in cell differentiation and proliferation.

Inappropriate activation or dysregulation of the Hedgehog pathway is believed to play a critical role in the proliferation and survival of certain cancer cells, including in basal cell carcinoma and medulloblastoma.

Known pathway-activating mutations include those that impair the ability of PTCH, a transporter-like Hh receptor, to restrain Smoothened (SMO) activation of transcriptional targets via the GLI family of latent transcription factors.

Binding of Hh ligand to PTCH is functionally equivalent to genetic loss of PTCH, in that pathway activation by either requires activity of SMO, a seven pass transmembrane protein that binds to and is inactivated by the pathway antagonist, cyclopamine.

The implication of PATCHED pathway activation in several cancer conditions, most notably in BCCs, has motivated much effort to set up experimental systems to assess the inhibitory activity of small molecules.

The existing systems to measure activation or inhibition of the activated SHH/PTCH pathway, are based on cell lines from human or mouse origin, or else genetically engineered mice to develop susceptibility toward spontaneous or provoked BCC development. With respect to cells, they schematically be classified in two categories destined to measure i-) endogenous cellular events after treatment; these events include triggering of a differentiation process and modulation of gene expression, notably of those genes known as transcriptional targets of pathway activation; ii-) cell lines engineered to report pathway activation/inhibition after transient or permanent introduction of reporter constructs made of responsive DNA driving a reporter gene; Cell lines developed so far are:
Human normal primary keratinocytes and fibroblasts in reconstructed skin where expression of GL1 and GLI2 mRNA have been measured to demonstrate inhibition by the small Robotnikinin molecule of SHH/PTCH pathway activation by SHH. (Stanton et al. 2009)
Healthy human primary keratinocytes from patients with nevoid basal cell carcinoma or Gorlin syndrome have been isolated to mimic the somatic loss of one PATCHED allele in sporadic BCC epidermal cells (Brellier et al., 2008a).

However, the above described cell lines have some disadvantages. Most of cell lines are not stable in the sense that after several passages the inserted genes expression decrease strongly or is shut down. Either those cell lines are not sufficiently robust to be efficient and sensitive to be used in a drug screening as a model. None of the cited prior art provides a system to allow a simple detection of activation in human epidermal keratinocytes.

On the other hand, animal models (mostly developed in the laboratory mouse) have been obtained by genetic engineering such as transgenesis of PTCH pathway activators (Gli1, Gli2, SHH, SMOM2) or abrogation of pathway inhibition by homologous recombinaison of the PATCHED 1 gene. In this respect, it is, however, worth noting that mouse skin is i-) very different from human skin (histology, DNA repair response, epidermal cell sternness) and ii-) never develop BCC along life, even after massive chronic ultraviolet irradiations, or chemical treatment such as the two hit DMBA/TPA carcinogenesis protocol. Thus, results of carcinogenesis obtained in mice are not necessarily transposable to human cells.

In this context, there is a clear need for developing a human cell line easy to produce and to use, for developing efficient and relevant model sensitive enough for the screening or assessment of molecules libraries in a context respecting 3-dimensional architecture of human skin. Organotypic skin culture models composed of those cancer permissive cells may fulfil these specifications in the short term (2-3 weeks). In addition, humanized mice obtained after human skin regeneration using the said cell line(s) may serve to long term assessment of anticancer drug screening with highly relevant potential assessment.

The inventors have developed a new cell line providing strong advantages with the latter respect. Indeed, the present invention provides an immortalized cell line of human keratinocytes with heterozygous mutation in the PATCHED1 gene (KNBCCS6 E6/E7). The cell line is stable overtime, particularly even after a high rate from passages in tissue culture.

In preferred embodiment of invention, the immortalized keratinocytes cell line according to the invention, express the PTCH1 mRNA and other members of the pathway necessary to appropriate activation and inhibition of the said SHH/PTCH pathway. Said cell line is immortalized by retroviral transduction of pLE6/E7SN. In addition, this cell line is produced in the absence of serum and without using feeder cells.

The invention provides also a process for obtaining immortalized human keratinocytes as describe above, comprising the following steps:
isolated human primary keratinocytes from healthy individual with Nevoid basal cell carcinoma syndrome (see below for the impact of the said pathway in human genetic and sporadic disease both of which include BCC susceptibility as a common clinical trait).
immortalized human primary keratinocytes from NBCCS patients by retroviral transduction with pLE6/E7SN
select cell line expression with a medium of selection

The invention provides also a drug screening method, wherein said immortalized keratinocytes cell line as described above is used to screen. In a preferred embodiment of the invention, the drug screening method comprises the following steps:
a). bringing one samples of organotypic skin cultures reconstituted using immortalized cell line as described in claim 1 into contact with one or more of the test compounds;
b). measuring histology of skin equivalent to assess development of epidermal invasion in the dermal equivalent (FIG. 3) or other conservative method after stable labeling of the said cell line using GFP as described (bergoglio 2007). By conservative method is meant that samples in vitro or in vivo, can be analysed in the real time without need of sample fixation and/or animal vector sacrifice (unless ethical rules should apply for sacrification of animal vectors).
c). selecting the compounds for which a modulation of epidermal invasion, is measured in b) and compared with no drug mixture. In a preferred embodiment, measurement of fluorescence emitted by GFP labeled cells in b) is used.

In a preferred embodiment, the drug identified and or selected according to the drug screening method as described above is an anti-tumor drug.

The present invention also provides an In vitro method for screening for candidate compounds for the preventive and/or curative treatment of cutaneous cancer and preferentially basal cell carcinoma, comprising the following steps:
a. bringing one samples of immortalized cell line as described above into contact with one or more of the test compounds or a mixture of compounds;
b. measuring the invasive properties of the said cell line,
c. selecting the compounds for which a modulation of invasion is measured in b) and compared with no drug mixture.

The invention regards also to a drug obtainable with the drug screening method as described above.

### DETAILED DESCRIPTION

Basal cell carcinomas (BCC) of the skin is the commonest human cancer. BCCs derive from epidermal keratinocytes. The great majority of BCCs occurs on photo-exposed skin due to ultraviolet induced mutagenesis. The steadily rising incidence of BCCs in the last decades is attributed to increasing enthusiasm for recreational sun exposure. Although BCC rarely metastasize, they can result in local destruction and invasion of underlying tissues and consequently, life threatening complications. BCC are usually treated by local surgical excision, topical chemotherapy, photodynamic therapy, but, according to tumor size, location and frequency, there may be considerable aesthetic sequalae. Thus, drawbacks of current BCC treatments strongly support the need for pharmacological innovations that should specifically target the SONIC in so far as inappropriate and constitutive activation of this pathway is associated with the vast majority of BCC (see below). Furthermore, molecules that target the SHH pathway could also be of interest in the treatment of other/non-BCC cancer conditions (or their stromal cells) (melanoma, pancreas, oeasophagus, liver, prostate, lung, muscle, colon) where the SONIC HEDGEHOG/PATCHED (see below) has also been found inappropriately activated (Scales and de Sauvage, 2009).

### The SHH/PATCHED Pathway

The SHH/PTCH signaling pathway is essential during embryogenesis and development where it controls cell fate by modulating proliferation and differentiation. Animal models, notably the fruit fly drosophila melanogaster, have shown that at specific stages of development, some cells produce and emit a signal, the Hedgehog molecule (HH), which, in turn, is received by target cells. In vertebrates, the family of Hedgehog molecules is composed of Sonic Hedgehog, SHH, Desert Hedgehog, DHH, and Indian Hedgehog, IHH. Target cells (of these ligands) express PATCHED (PTCH), a putative twelve pass transmembrane protein acting as the receptor of HH molecules. When HH molecules are not expressed and/or not secreted at the vicinity of target cells, PTCH acts as a repressor of the pathway by inhibiting another transmembrane protein called SMOTHENED (SMO). SMO is a putative seven pass transmembrane protein apparented to G-protein coupled receptors. The inhibition of SMO by PTCH is relieved in the presence of HH molecules bound to PTCH. De-repression of SMO leads to activation of transcription factors of the GLI family (named GLI 1, 2 and 3) that activate (GLI1 and 2) or repress (GLI3), the transcription of their target genes. Interestingly, PTCH1 is a transcriptional target of GLI1 and GLI2 factors.

The importance of the SHH/PTCH pathway is illustrated by severe diseases due to mutations affecting its integrity at different levels. Notably, in the human, heterozygous mutations in the PTCH1 gene are responsible for the dominantly inherited genetic syndrome called nevoid basal cell carcinoma syndrome (NBCCS or Gorlin syndrome). NBCCS patients are highly prone to BCCs that generally (about 50% cases) present with a loss of heterozygosity in the PTCH1 locus. In Gorlin patients, more than 50% BCCs also bear mutation in the tumor suppressor gene TP53, suggesting some cooperation of the P53 and the SHH/PTCH pathways toward development of BCCs. Very interestingly, the two PTCH1 alleles are also lost in most sporadic (general population) BCCs; in the latter case, again, the two TP53 alleles are found mutated in 10 to 50% sporadic BCCs. 20-30% sporadic BCCs are mutated in both TP53 and PTCH1 (Soufir et al., 2006). In both NBCSS and sporadic BCCs, inactivation of PTCH results in constitutive activation of the pathway with accumulation GLI1 and GLI2 mRNAs.

The Implication of the SHH/PTCH pathway activation in several cancer conditions, most notably in BCCs, has motivated much effort to set up experimental systems to assess the inhibitory activity of small molecules.

The existing systems of activity measure are based on cell lines from human or mouse origin. Theses cells can schematically be classified in two categories destined to measure i-) endogenous cellular events after treatment; these events include triggering of a differentiation process and modulation of gene expression, notably of those genes known as transcriptional targets of pathway activation; ii-) cell lines engineered to report pathway activation/inhibition after transient or permanent introduction of reporter constructs made of responsive DNA driving a reporter gene. Cell lines developed so far in the prior art reveals that none of those system allows simple detection of activation in human epidermal keratinocytes in a context that reproduces 3-dimensional architecture of skin in the respect of dermo-epidermal interactions. As underlined above, models of genetically engineered mice are not necessarily relevant of human skin and cancer and hence assessment of relevant treatments to be applied thereof.

To provide a simple detection system, the inventors have worked to develop a human cell line in the respect of the following specifications (i.e. what we need for easy, efficient, relevant, sensitive assessment of molecules libraries):
human cells.
epidermal cells,
growth in standard medium,
needing no feeders,
stably transformed, by immortalizing antigen (E6/E7 from HPV 16)

To fulfil these specifications, the strategy was to use a human cell strain derived from normal human epidermis (Otto et al., 1999). Concerning the easiness of growth and the independency toward feeder cells, we decided to abrogate or at least to attenuate, the expression of the tumor suppressor gene TP53. Indeed, it is known for instance, that HaCat cells (that derive form an epidermal carcinoma with the two TP53 allele mutated (Lehman et al., 1993)), are capable of growing in the absence of feeders cells. However, as explained below, use of these cells is not necessarily appropriate to measure pathway activation.

Previous work from the laboratory showed that P53 stabilisation after a single UVB irradiation is higher and prolonged in NBCCS compared to control keratinocytes (Brellier et al., 2008b). Also, report by Stecca and Ruiz-i-Altaba revealed mutual inhibition of Gli1 and P53 (Stecca and Ruiz i Altaba, 2009). Together with molecular epidemiology studies (showing mutations in both TP53 and PATCHED, in 20-30% BCCs), these results convergently indicate interaction between the P53 and the SHH/PTCH pathways (Soufir et al., 2006).

Thus, it is reasoned that abrogation or attenuation of the P53 pathway using E6-E7 oncogenic proteins of Human Papilloma Virus 16 (HPV16) would favor activation of the SHH/PTCH pathway in human epidermal keratinocytes. It must also be emphasized that, although they are generally mutated in TP53, keratinocytes from squamous cell carcinoma (SCC) are never mutated in PTCH1 or at least do not show activation of the pathway (accumulation of target genes transcripts). This observation suggest that the SHH/PTCH pathway is not active in cells at the origin of SCCs (a subpopulation of epidermal stem cells). In spite of their easy growth in culture, SCC cells (such as HaCat cells) would thus not constitute appropriate recipient cells for reporter constructs. Preferably, it was decided to use human primary keratinocytes transformed with the E6-E7 oncogenic proteins.

Thus, the present invention provides an immortalized cell line of human keratinocytes selected for specific growth properties and invasiveness in 3D culture conditions.

The immortalized keratinocyte cell line of according to the invention expresses the PTCH protein. Said cell line is immortalized by retroviral transduction. The skilled in the art is familiar with retroviral transduction techniques and all of them are applicable to the present invention. Any kind of retrovirus can be used such as Moloney murine leukemia virus (MoMLV), lentivirus, Eptein-Barr virus (EBV). MoMLV is preferred for high performance of infection in human primary keratinocytes (Bergoglio et al., 2007). The retroviral transduction of pLE6/E7SN is preferred in this context.

In addition, our previous observations have indicated that primary NBCCS primary fibroblasts isolated from healthy skin expressed a transcriptome resembling that characterized in fibroblasts associated to sporadic carcinomas (CAF) (Valin et al., 2009; Valin and Magnaldo, 2008). Together, these observations strongly support the idea of a strong contribution of dermal fibroblasts in carcinoma development in NBCCS patients. Thus, in the present invention, skin permissive to epidermal invasion is reconstituted using the said immortalized keratinocytes cell line overvaying a dermal equivalent composed of NBCCS fibroblats.

In addition, this cell line is produced in the absence of serum and without using feeder cells which provides a greater benefit in terms of feeding and growth time. Indeed, serum or feeder cells can incorporate or secrete substances which can by their presence interfere or modify the activity response. Cells are grown in a definite medium providing the advantage of growing cells in medium which does not interfere with activity response. The invention provides thus a robust model with expected or calibrated response which avoids any interfering factors.

The invention provides also a drug screening method, wherein said immortalized keratinocytes cell line as described above is used to screen. The invention relates to an in vitro/in vivo screening method of PTCH pathway inhibitors for treating skin cancer and preferably BCC, comprising determining the capacity of said drug to inhibit or down regulate expression or biological activity of PTCH.

In a preferred embodiment of the invention, the drug screening method comprises the following steps:
a). bringing one samples of immortalized cell line as described above into contact with one or more of the test compounds;
b). measuring the invasiveness of the immortalized keratinocytes cell line as described above
c). selecting the compounds for which a modulation of invasiveness, is measured in
b) and compared with no drug mixture.

In the context of the invention, cells are labelled with GFP, GFP derivatives, luciferase etc., reported with fluorescence or bioluminescence methods known by the skilled artisan. In a preferred embodiment, the reporter/tracer gene is GFP.

The present invention provides tools for selecting SHH/PTCH pathway modulators. Those modulators are activators or inhibitors.

In a preferred embodiment, the drug identified and/or selected according to the drug screening method as described above is an anti-tumor drug. Inhibition efficacy of one or several drug candidates (isolated or in a mixture) preferably with increasing concentration, is assessed by histology or measure of reporter activity/tracer gene intensity of activity. The examples provide an illustration with a particular embodiment in histological reporter system (FIG. 5).

In another embodiment, the present invention provides an in vivo tool for assessing keratinocyte invasiveness in a humanised animal model (such as mouse or mini-pig) having said KNBCCS6 E6/E7 immortalised cell line to follow activity of drug candidate kinetics. The in situ assessment provides a considerable advantage with the GFP or derivatives fluorescence or any other reporter/tracer gene as the effect of drug candidate is eye visible and does not require further read out methods, nor it does require ending up of experiments including sample processing and vector animal sacrification. This tool is also usable with reconstructed epidermis in vitro or skin regenerated in vivo.

The examples that follow will illustrate the invention without limiting the scope thereof.

### EXAMPLES

### Materials and Methods

### Cell Culture

Human primary keratinocytes (named KNBCSS6 E6/E7) were isolated from a healthy non photo-exposed skin biopsy of NBCCS patient as described (Otto et al., 1999)

### Cell Transformation and Selection

The NBCCS6 keratinocyte cell line was immortalized by retroviral transduction with pLE6/E7SN resulting in CTRL and NBCCS-E6-E7 (FIG. 1) (Halbert et al., 1991, 1992). Cells were grown at 37° C. in a humified atmosphere containing 5% CO2, DMEM medium, 50 U/ml penicillin, 50 μg/ml streptomycin, 0.125 μg/ml amphotericin B, 2 mM L-Glutamine, 1 mM Sodium pyruvate, 1× non essential amino acids.

G418 is an aminoglycoside antibiotic similar in structure to gentamicin B1, produced by Micromonospora rhodorangea. G418 blocks polypeptide synthesis by inhibiting the elongation step in both prokaryotic and eukaryotic cells. Resistance to G418 is conferred by the Neomycin resistance gene (neo) from Tn5 encoding an aminoglycoside 3′-phosphotransferase, APH 3′II.

Selection in mammalian cells is usually achieved in three to seven days with concentrations ranging from 200 to 1000 μg/ml (Amaudeau-Bégard et al., 2003).

KNBCCS6 E6/E7 immortalized keratinocytes were grown at 37° C. in a humified atmosphere containing 5% CO2. Serum free medium (SFM Gibco ref: 10725-018), contained 0.1 mM CaCl₂, 2.10⁻⁴ ng/ml EGF, 50 U/ml penicillin, 50 μg/ml streptomycin, 0.125 μg/ml amphotericin B.

NBCC6 primary fibroblast cells were grown at 37° C. in a humified atmosphere containing 5% CO2. DMEM medium, 50 U/ml penicillin, 50 μg/ml streptomycin, 0.125 μg/ml amphotericin B, 2 mM L-Glutamine, 1 mM Sodium pyruvate, 1× non essential amino acids.

### Small Molecules Modulators of the SHH/PTCH Pathway

Purmorphamine (SMO agonist) and GDC-0449 (SMO antagonist) were diluted in DMSO at stock concentrations of 50 mM and 10 mM, respectively. To avoid side effects and toxicity, the final concentration of DMSO was fixed to 0.1% DMSO.

Transformation of the KNBCC6 Keratinocyte Cell Line by the E6/E7 Oncoproteins

Effective transformation of the KNBCCS6 cell line was assessed by analysing its growth properties and attenuation of expression of the P53 tumor suppressor protein. FIG. 2 shows a western blot analysis of P53 in cell extracts prepared from preconconfluent (about 80%) primary epidermal keratinocytes (KNBCCS6), primary epidermal keratinocytes (KNBCCS6) after transformation using the E6/E7 encoding retroviral vector (KNBCCS6 E6/E7), The western blot shows the drastic decrease in the amount of the P53 protein.

Organotypic skin reconstruction using KNBCCS E6/E7 Immortalized Keratinocytes Overlaying a Dermal Equivalent Containing Autologous NBCCS Primary Fibroblasts:

Organotypic skin reconstruction using KNBCCS E6/E7 was performed using standard protocols as described in (Bemerd et al., 2001). In brief 10⁸ NBCCS6 primary fibroblasts cultured in standard culture medium as described above were embedded in a bovine collagen I lattice. After contraction (48 hrs) lattices were fixed on plastic using a collagen glue. KNBCCS6 were then seeded (50 000 cells per lattice) within a stainless ring, left for 6 days in immersion; the day 6) before emersion (day 7), mounts were treated with appropriate concentration of SHH:PTCH pathway inhibitor with a final a 0.1% DMSO concentration until the end of experiment. Control cultures were treated with 0.1% DMSO. After 7 days of emersion at air-liquid interface, cultures were harvested for subsequent analyses including histology and assessment of tacer/reporter gene activity. (FIG. 3 and FIG. 4) FIG. 3 depicts the organotypic skin reconstruction showing invasive properties of KNBCC6 E6/E7 cell line over a dermal equivalent composed of autologous NBCCS6 primary fibroblasts

FIGS. 4 A, B & C depicts an organotypic skin reconstruction and effect of SHH/PTCH inhibitor and in particular FIG. 4 A, shows the same organotypic skin cultures system as in FIG. 3 composed of an epidermis developed from KNBCCS6 E6/E7 immortalized keratinocyte overlaying a dermal equivalent containing autologous NBCCS6 primary fibroblasts. 4 fields are shown.

FIGS. 4B, 4C show the same as FIG. 4A, except that organotypic skin cultures were grown 24 hrs before being raised at air-liquid interface in the presence of 10 mM of GDC-0449 (B) or 10 mM (SANT1) SHH/PTCH pathway inhibitors. Note that the size and frequency of epidermal invasion were substantially decreased in the presence of either of both inhibitors.

### Conclusions

We have developed an immortalized human cell line of keratinocytes that respond to activation by an agonist (purmorphamine) and inhibition of the activation by an antagonist (GDC-0449, SANT1) of the SHH/PTCH signaling pathway.

The KNBCCS6 cell line does not need to be cultured in the presence of feeder cells. Theses cells can be expanded in a simple defined medium and they can grow easily due to immortalization. This tool is 3D organotypic culture system to evaluate the efficacy of anti-tumoral molecules specifically targeting inappropriate activation of the SHH/PTCH pathway occurring in BCC but also in numerous non BCC cancers including other cancer implicating inappropriate activation of the SHH/PTCH pathway. Assessment of inhibitor efficacy is applicable in the long term by skin regeneration in vivo using animal vector including the mouse or the mini pig systems.

### FIGURES

FIG. 1: Schematic map of the LE6E7 SN proviral construct. E6E7, sequence of the human papilloma virus 16 encoding the E6 and E7 transforming proteins.

FIG. 2: western blot analysis of the expression of the P53 protein in the indicated cells. GAPDH is a control of loading attenting that similar amount of protein is present in each lane.

FIG. 3: Organotypic skin reconstruction showing invasive properties of KNBCC6 E6/E7 cell line over a dermal equivalent composed of autologous NBCCS6 primary fibroblasts

FIG. 4: Organotypic skin reconstruction and effect of SHH/PTCH inhibitor

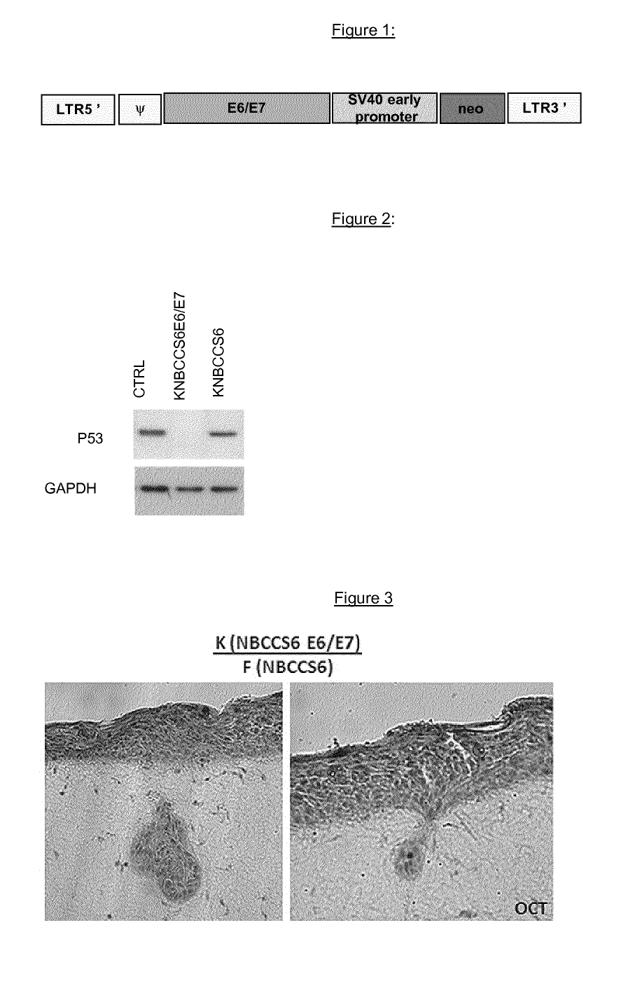

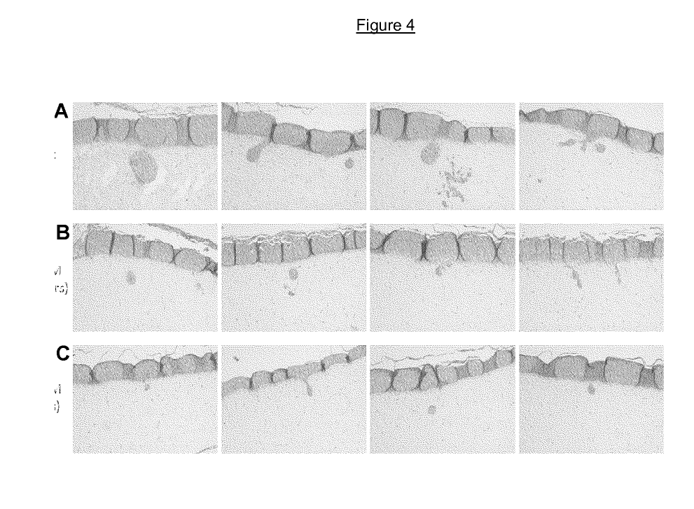

### REFERENCES

Amaudeau-Bégard. C., Brellier, F., Chevallier-Lagente, O. Hoeijmakers, J. H., Bernerd, F., Sarasin, A., and Magnaldo, T. (2003). Genetic correction of DNA repair deficient/cancer prone xeroderma pigmentosum group C keratinocytes. Hum Gene Ther 14, 983-996.
Bergoglio, V., Larcher, F., Chevallier-Lagente, O., Bernheim, A., Danos, O., Sarasin, A., Rio, M. D., and Magnaldo, T. (2007). Safe selection of genetically manipulated human primary keratinocytes with very high growth potential using CD24. Mol Ther 15, 2186-2193.
Bernerd, F., Asselineau, D., Vioux, C., Chevallier-Lagente, O., Bouadjar, B., Sarasin, A., and Magnaldo. T. (2001). Clues to epidermal cancer proneness revealed by reconstruction of DNA repair-deficient xeroderma pigmentosum skin in vitro. Proc Natl Acad Sci USA 98, 7817-7822.
Brellier, F., Bergoglio, V., Valin, A., Barnay, S., Chevallier-Lagente, O., Vielh, P., Spatz, A., Gorry. P., Avril, M. F., and Magnaldo, T. (2008a). Heterozygous mutations in the tumor suppressor gene PATCHED provoke basal cell carcinoma-like features in human organotypic skin cultures. Oncogene 27, 6601-6606.
Brellier, F., Valin, A., Chevallier-Lagente, O., Gorry, P., Avril, M. F., and Magnaldo, T. (2008b). Ultraviolet responses of Gorlin syndrome primary skin cells. Br J Dermatol 159, 445-452.
Halbert, C. L., Demers, G. W., and Galloway, D. A. (1991). The E7 gene of human papillomavirus type 16 is sufficient for immortalization of human epithelial cells. J Virol 65, 473-478.
Halbert, C. L., Demers, G. W. and Galloway, D. A. (1992). The E6 and E7 genes of human papillomavirus type 6 have weak immortalizing activity in human epithelial cells. J Virol 66, 2125-2134.
Lehman, T. A., Modali, R., Boukamp, P., Stanek, J., Bennett, W. P., Welsh, J. A., Metcalf, R. A., Stampfer, M. R., Fusenig, N., Rogan, E. M., et al. (1993). p53 mutations in human immortalized epithelial cell lines. Carcinogenesis 14, 833-839.
Otto, A. I., Riou, L., Marionnet. C., Mori, T., Sarasin, A., and Magnaldo, T. (1999). Differential behaviors toward ultraviolet A and B radiation of fibroblasts and keratinocytes from normal and DNA-repair-deficient patients. Cancer Res 59, 1212-1218.
Scales, S. J., and de Sauvage, F. J. (2009). Mechanisms of Hedgehog pathway activation in cancer and implications for therapy. Trends Pharmacol Sci 30, 303-312.
Soufir, N., Gerard, B., Portela, M., Brice, A., Liboutet, M., Saiag. P., Descamps, V., Kerob, D., Wolkenstein, P., Gorin, I., et al. (2006). PTCH mutations and deletions in patients with typical nevoid basal cell carcinoma syndrome and in patients with a suspected genetic predisposition to basal cell carcinoma: a French study. Br J Cancer 95, 548-553.
Stecca, B. and Ruiz i Altaba, A. (2009). A GLI1-p53 inhibitory loop controls neural stem cell and tumour cell numbers. EMBO J 28, 663-676.
Valin, A., Barnay-Verdier, S., Robert, T., Ripoche, H., Brellier, F., Chevallier-Lagente, O., Avril, M. F., and Magnaldo, T. (2009). PTCH1+/− dermal fibroblasts isolated from healthy skin of Gorlin syndrome patients exhibit features of carcinoma associated fibroblasts. PLoS One 4, e4818.
Valin, A., and Magnaldo, T. (2008). Method for determining a predisposition to basal cell carcinoma and for screening treatments thereof. Brevet européen, déposé le 05 Aout 2008, EP 08 3054486.

1. An immortalized cell line of human keratinocytes with a heterozygous mutation in the PATCHED1 gene (KNBCCS6 E6/E7) that has stable expression overtime, even after a high rate from passages in tissue culture.

2. The immortalized cell line of claim 1, wherein the cell line is immortalized by retroviral transduction of pLE6/E7SN.

3. The immortalized cell line of claim 1, wherein the cell line is produced in the absence of serum or without using feeder cells.

4. The immortalized cell line of claim 3, wherein the cell line expresses PTCH 1 mRNA.

5. The immortalized cell line of claim 4, wherein the cell line expresses the other members of the pathway SHH/PTCH necessary to appropriately inhibit and activate the pathway.

6. A process for obtaining the immortalized human keratinocytes of claim 1, the process comprising the following steps:
isolating human primary keratinocytes from a healthy individual having Nevoid Basal Cell Carcinoma Syndrome (NBCCS),
immortalizing human primary keratinocytes from NBCCS patients by retroviral transduction with pLE6/E7SN, and
selecting a cell line expression with a medium of selection.

7. A drug screening method, the method comprising screening for a drug using the immortalized cell line of claim 1.

8. The drug screening method of claim 7, the method comprising the following steps of:
a. bringing one sample of organotypic skin cultures reconstituted using the immortalized cell line of claim 1 into contact with one or more test compounds or with a mixture of compounds,
b. measuring histology of a skin equivalent to assess development of epidermal invasion in the skin equivalent or other conservative method after stable labeling of the cell line using GFP, and
c. selecting the compounds for which a modulation of epidermal invasion is measured in step b) when compared to the epidermal invasion in the absence of any test compounds or mixture of compounds.

9. The drug screening method of claim 8, wherein samples are analyzed in vitro or in vivo in real time without need of sample fixation and/or animal vector sacrifice.

10. The drug screening method of claim 7, wherein the identified drug is an antitumor drug.

11. An In vitro method for screening candidate compounds for preventive and/or curative treatment of cutaneous cancer, the method comprising the following steps of:
a. bringing one sample of the immortalized cell line of claim 1 into contact with one or more test compounds or with a mixture of compounds,
b. measuring invasive properties of the cell line, and
c. selecting the compounds for which a modulation of epidermal invasion is measured in step b) when compared to epidermal invasion in the absence of any test compounds or mixture of compounds.

12. A drug identified by the drug screening method of claim 7.

13. Organotypic skin cultures, reconstituted using the immortalized cell line of claim 1, overlaying a dermal equivalent containing autologous NBCCS6 primary keratinocytes.
